(19) United States
(12) Patent Application Publication
(10) Pub. No.: US 2010/0215644 A1
(43) Pub. Date: Aug. 26, 2010

(54) ANALYSIS OF NODES IN CELLULAR PATHWAYS

(75) Inventors: Wendy J. Fantl, San Francisco, CA (US); Ying-Wen Huang, Palo Alto, CA (US)

Correspondence Address: WILSON, SONSINI, GOODRICH & ROSATI / NODALITY, INC, 650 Page Mill Road, Palo Alto, CA, 94304-1050, US

(73) Assignee: Nodality, Inc. A Delaware Corporation

(21) Appl. No.: 12/713,165

(22) Filed: Feb. 25, 2010

Related U.S. Application Data
(60) Provisional application No. 61/155,373, filed on Feb. 25, 2009.
(60) Provisional application No. 61/177,935, filed on May 13, 2009.
(60) Provisional application No. 61/182,638, filed on May 29, 2009.
(60) Provisional application No. 61/240,193, filed on Sep. 5, 2009.

(51) Int. Cl. A61K 39/395; C12Q 1/68; G01N 33/53; C12Q 1/48; C12Q 1/37; C12Q 1/02; A61K 38/16; A61K 31/7088

(52) U.S. Cl. 424/130.1; 435/6; 435/7.1; 435/15; 435/23; 435/29; 514/12; 514/44 R

(57) ABSTRACT

An embodiment of the present invention is a method for measuring activity of cell pathways, such as the cell cycle pathway and correlating the resulting profile to phenotypes. The resulting correlations are useful in diagnosis, prognosis, selection and development of drug treatment regimens, and drug screening applications.

### CROSS-REFERENCE

This application claims the benefit of U.S. Provisional Applications No. 60/155,373, filed Feb. 25, 2009, Provisional Application No. 61/177,935, filed on May 13, 2009, Provisional Application 61/182,638, Filed on May 29, 2009, and Provisional Application No. 61/240,193, filed on Sep. 5, 2009, which applications are incorporated herein by reference.

### BACKGROUND OF THE INVENTION

Many conditions are characterized by disruptions of cellular pathways that lead, to aberrant control of cellular processes that may result in uncontrolled growth and increased cell survival. These disruptions are often caused by changes in the activity of molecules participating in cellular pathways. For example, alterations in specific signaling pathways have been described for many cancers.

Elucidation of the signal-transduction networks that drive neoplastic transformation in both solid tumors and hematological malignancies has led to rationally designed cancer therapeutics that target signaling molecules. Accordingly, there is a need to look at single cells and/or cell populations to determine what signaling events may contribute to their responses to compounds.

This application claims the benefit of U.S. Provisional Applications No. 60/155,373, filed Feb. 25, 2009, Provisional Application No. 61/177,935, filed on May 13, 2009, Provisional Application 61/182,638, Filed on May 29, 2009, and Provisional Application No. 61/240,193, filed on Sep. 5, 2009, which applications are incorporated herein by reference.

### SUMMARY OF THE INVENTION

One embodiment of the invention measures nodes in cellular pathways, such as the cell cycle. It is useful to understand the effect of compounds and other modulators on cell cycle progression and apoptosis and the present invention presents an embodiment that can make that determination. Knowledge of the cellular pathway can impact several health care issues, such as drug development, therapeutic treatment development, therapeutic treatment selection, patient management, diagnosis, as well as analyzing the mechanism by which a cell, such as a tumor cell, may change and adapt under therapeutic pressure.

One embodiment of the present invention discloses ways of using fluorescent detection of a phosphorylated substrate, termed phosphoflow to assist in the analysis. One method that will be useful is multiparametric phosphoflow technology which can monitor multiple pathways simultaneously within heterogeneous cell populations at the single cell level. Other methods which allow the researcher to detect multiple signaling pathways will also be useful.

In one or more of the following non-limiting embodiments, the present invention can be achieved by performing the active steps below and correlating observations of pathway activity with a phenotype. Drugs or any other modulator, such as a biologically active moledule, can be evaluated for therapeutic activity, dosing, schedule, efficacy, and a diagnosis or prognosis can be made.

One embodiment of the invention involves methods for monitoring response of cancer to a drug, such as a drug specifically designed to correct the molecular abnormalities that may underlie a cancer phenotype. Some methods can be useful to select dose and/or scheduling of these drugs in patients.

One embodiment of the invention is a method to identify proliferating cells by measuring components of the cell cycle that indicate cell proliferation. Another embodiment is a method for drug development that may address “on” or “off” target activity of a compound. Another embodiment of the invention is useful in patient selection in order to determine the likelihood of a patient to respond to a therapeutic based on the number of cycling cells in a specimen. Specimens can include bone marrow, peripheral blood, biopsy fine needle aspirates, circulating tumor cells, and the like. Another embodiment of the invention is a method for detecting a combination of therapeutic agents that may inhibit cell proliferation.

### BRIEF DESCRIPTION OF THE DRAWINGS

FIG. 1 shows the identification of sub-populations of cells in bone marrow mononuclear cells (BMMCs) from MDS patients.

FIG. 2 shows that ON01910.Na induces arrest in G2/M and cell death.

FIG. 3 shows that ON01910.Na induces cell death in erythroblasts as determined by scatter properties.

FIG. 4 shows that ON01910.Na mediates dephosphorylation of p-Cdk1^{Y15} in erythroblasts.

FIG. 5 shows that ON01910.Na mediates an increase in p-HistoneH3^{S28} in erythroblasts.

FIG. 6 shows that ON01910.Na mediates an increase in cyclin B1 in erythroblasts.

FIG. 7 shows the effect of ON01910.Na titration on cyclin B1 expression, p-Cdk1^{Y15} levels, and p-HistoneH3^{S28} levels.

FIG. 8 shows two-dimensional multiparametric cell cycle analysis of p-Cdk1^{Y15}, p-Histone H3^{S28}, and cyclin B1 levels following ON01910.Na treatment.

FIG. 9 shows effect of Vidaza® cytidine analog and Dacogen® cytidine analog on the cell cycle.

FIG. 10 shows that Vidaza® cytidine analog mediates a decrease in DNMT1.

FIG. 11 shows a dose dependent decrease of cycling bone marrow mononuclear cells as measured by p-Cdk1^{Y15} decrease following ON01910.Na titration.

FIG. 12 shows that ON01910.Na does not significantly alter viability of healthy bone marrow mononuclear cells.

FIG. 13 shows simultaneous changes in cell cycle signaling molecules at the same drug concentration in U937 cells following ON01910.Na titration.

### DETAILED DESCRIPTION OF THE INVENTION

The present invention incorporates information disclosed in other applications and texts. The following publications are hereby incorporated by reference in their entireties: Haskell et al, Cancer Treatment, 5^{th} Ed., W.B. Saunders and Co., 2001; Alberts et al., Molecular Biology of The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996; and Immunophenotyping, Chapter 9: Use of Multiparameter Flow Cytometry and Immunophenotyping for the Diagnosis and Classification of Acute Myeloid Leukemia, Stelzer, et al., Wiley, 2000.

Patents and applications that are also incorporated by reference in their entirety include U.S. Pat. Nos. 7,381,535 and 7,393,656 and U.S. patent application Ser. Nos. 10/193,462; 11/655,785; 11/655,789; 11/655,821; 11/338,957, 61/048,886; 61/048,920; and 61/048,657.

Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7.

Relevant articles include: Krutzik et al., High-content single-cell drug screening with phosphospecific flow cytometry, Nat. Chem. Biol., Dec. 23, 2007, 4(2): 132-142; Irish et al., Flt3 Y591 duplication and Bcl-2 over expression are detected in acute myeloid leukemia cells with high levels of phosphorylated wild-type p53, Blood, Mar. 15, 2007, 109(6): 2589-96; Irish et al. Mapping normal and cancer cell signaling networks: towards single-cell proteomics, Nat. Rev. Cancer, February 2006, 6(2): 146-155; Irish et al., Single cell profiling of potentiated phospho-protein networks in cancer cells, Cell, Jul. 23, 2004, 118(2): 217-228; Schulz, K. R., et al., Single-cell phospho-protein analysis by flow cytometry, Curr. Protoc. Immunol., August 2007, 78:8 8.17.1-20; Krutzik, P. O., et al., Coordinate analysis of murine immune cell surface markers and intracellular phosphoproteins by flow cytometry, J. Immunol., Aug. 15, 2005, 175(4): 2357-65; Krutzik, P. O., et al., Characterization of the murine immunological signaling network with phosphospecific flow cytometry, J. Immunol., Aug. 15, 2005, 175(4): 2366-73; Shulz et al., Curr. Prot. Immun., 2007, 78:8.17.1-20; Krutzik, P. O. and Nolan, G. P., Intracellular phospho-protein staining techniques for flow cytometry: monitoring single cell signaling events, Cytometry A, Sep. 17, 2003, 55(2): 61-70; Hanahan D., Weinberg, The Hallmarks of Cancer, Cell, Jan. 7, 2000, 100(1): 57-70; and Krutzik et al, High content single cell drug screening with phosphospecific flow cytometry, Nat. Chem. Biol., February 2008, 4(2): 132-42; Marcos Malumbes & Mariano Barbacid, Cell Cycle, CDKs, and Cancer: A Changing Paradigm, 9 Nature Rev. Cancer 153 (2009); Gary K. Schwartz & Manish A. Shah, Targeting the Cell Cycle: A New Approach to Cancer Therapy, 23 J. Clinical Oncol. 9408 (2005). Experimental and process protocols and other helpful information can be found at http://proteomics.stanford.edu. The articles and other references cited below are also incorporated by reference in their entireties for all purposes.

The discussion below describes some of the preferred embodiments with respect to particular diseases. However, it should be appreciated that the principles may be useful for the analysis of many other diseases as well. Without being limited, example diseases include cancers, autoimmune diseases, metabolic disorders, degenerative/wasting diseases, neurological diseases. For example, cancers can include solid tumors such as glioblastoma, colon, breast, thyroid, ovarian, prostate, lung, melanoma and pancreatic cancers and blood cancers such as AML, MDS, ALL, CLL and CML. See Hanahan D., Weinberg, The Hallmarks of Cancer, Cell, Jan. 7, 2000, 100(1): 57-70 cited above. Other examples are shown in Wood et al, The Genomic Landscapes of Human Breast and Colorectal Cancers. Science (2007) 318: 1108-1113; Jones et al., Core Signaling Pathways in Human Pancreatic Cancers Revealed by Global Genomic Analyses. Science (2008) 321: 1801-1806; and Parsons et al., An Integrated Genomic Analysis of Human Glioblastoma Multiforme. Science (2008) 321: 1807-1812 which are all incorporated by reference in their entireties.

In some embodiments, the methods of the present invention are useful for monitoring the efficacy of drugs directly, by looking at pathways in affected cells, or by using other cells as a surrogate.

### General Methods

The following will discuss research and diagnostic methods, instruments, reagents, kits, and the biology involved in analyzing cell cycle and apoptotic pathways. One aspect of the invention involves contacting a cell with at least one of a plurality of compounds; and analyzing at least one activation state of at least one activatable element or node using techniques known in the art, such as phosphoflow cytometry, where one or more individual cells can be simultaneously analyzed for one or more characteristics.

In some embodiments, the present invention is directed to select at least one of a plurality of compounds for efficacy in modulating a pathway, such as for optimization and preclinical studies. In some embodiments, the present invention is directed to determining dosing and scheduling of at least one of a plurality of compounds that can be used to treat a subject. In some embodiments, the invention employs techniques, such as flow cytometry, imaging approaches, mass spectrometry based flow cytometry, nucleic acid microarrays, or other phenotypic assays.

In some embodiments, the invention is directed to methods for determining the activation level of one or more activatable elements in a cell upon administration with one or more modulators. The activation of an activatable element in the cell upon administration with one or more modulators can reveal operative pathways in a condition that can then be used, e.g., as an indicator to predict a course of the condition, identify a risk group, predict an increased risk of developing secondary complications, choose a therapy for an individual, predict response to a therapy for an individual, determine the efficacy of a therapy in an individual, and determine a clinical outcome for an individual. In some embodiments, the activation level can be compared to another cell contacted with one or more modulators. In some embodiments, this comparison can be used to determine the presence or absence of a change in the activation level of the activatable element. In some embodiments, the comparison to another cell uses a normal cell for the comparison. In some embodiments, the modulator or modulators used can be a targeted cell cycle pathway modulator, as further described below.

In some embodiments, the invention is directed to methods of determining a phenotypic profile of a population of cells by exposing the population of cells to a plurality of modulators in separate cultures, wherein at least one of the modulators is an inhibitor, determining the presence or absence of an increase in activation level of an activatable element in the cell population from each of the separate culture and classifying the cell population based on the presence or absence of the increase in the activation of the activatable element from each of the separate culture.

One or more cells or cell types, or samples containing one or more cells or cell types, can be isolated from body samples. The cells can be separated from body samples by centrifugation, elutriation, density gradient separation, apheresis, affinity selection, panning, FACS, centrifugation with Hypaque, and the like. By using antibodies specific for markers expressed by particular cell types, a relatively homogeneous population of cells may be obtained. Alternatively, a heterogeneous cell population can be used. Cells can also be separated by using filters. For example, whole blood can also be applied to filters that are engineered to contain pore sizes that select for the desired cell type or class. Peripheral blood mononuclear cells (PBMCs) and bone marrow mononuclear cells (BMMCs) may be used. Rare cells can be filtered out of diluted, whole blood following the lysis of red blood cells by using filters with pore sizes between 5 to 10 μm, as disclosed in U.S. patent application Ser. No. 09/790,673. Rare cells may then be used in any method described herein. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Patent Application Nos. 61/048,886; 61/048,920; and 61/048,657. Exemplary established cell lines may also be used, such as (for hematological tumors) U937, THP, Kg-1, OPM2, MM1, TF-1, and ESM; (for solid tumors) U87Mg, PC3, BT474, WI-38, and A549. See also, the commercial products from companies such as BD and BCI as identified above.

See also U.S. Pat. Nos. 7,381,535 and 7,393,656. All of the above patents and applications are incorporated by reference as stated above.

The term “patient” or “individual” as used herein includes humans as well as other mammals. The methods generally involve determining the status of an activatable element. The methods also involve determining the status of a plurality of activatable elements.

The analysis of a cell and the determination of the status of an activatable element can comprise classifying a cell as a cell that is correlated to a patient response to a treatment. In some embodiments, the patient response is selected from the group consisting of complete response, partial response, nodular partial response, no response, progressive disease, stable disease and adverse reaction.

The classification of a rare cell according to the status of an activatable element can comprise classifying the cell as a cell that can be correlated with minimal residual disease or emerging resistance. See U.S. Application No. 61/048,886, which is incorporated by reference.

In some embodiments, the invention is a method of classification of a cell or a population of cells by measurement of one or more activatable elements of the cell cycle pathway. These activatable elements can be, for example, cyclin or cyclin dependent kinase (cdk) proteins, such as cyclin A, cyclin B, cycline B1, cyclin D, cyclin E, CDK1, CDK2, CDK3, CDK4, CDK5, CDK6, CDK7, CDK8, CDK9, CDK10, CDK11, CDK12, CDK13; regulators of cyclin-cdk complexes, such as Wee, CDK-activating kinase (CAK), Cdc20 and Cdc25; retinoblastoma susceptibility protein (Rb); cell cycle inhibitor proteins, such as cip/kip family proteins, such as p21, p27, p57; p53; Tumor Growth Factor beta (TGFβ); INK4a/ARF family proteins such as p16INK4a and p14ARF. Other cell cycle pathway activatable elements include, but are not limited to, Plk1, Histone H3, caspase-2, caspase-3, caspase-6, caspase-7, caspase-8, caspase-9, cytochrome c, Bcl-2, survivin, Xiap, PARP, Chk1, Chk2, histone 2AX, TRADD, FADD, Fas receptor, FasL, caspase-10, BAX, BID, BAK, BAD, Bcl-X_{L}, SMAC, VDAC2, Bim, Mcl-1 and AIF. Any one or more of these proteins can be used to characterize one or more cells having a cell cycle disorder, or be used to determine the efficacy of one or more modulators (such as inhibitors) of the cell cycle pathway, using methods described below.

The classification of a cell according to the status of an activatable element can comprise selecting a method of treatment. Examples of treatment methods include, but are not limited to, compounds that control some of the symptoms of a condition, such as aspirin and antihistamines, compounds that stimulate red blood cell production, such as erythropoietin or darbepoietin, compounds that reduce platelet production, such as hydroxyurea, anagrelide, and interferon-alpha, compounds that increase white blood cell production, such as G-CSF, chemotherapy, biological therapy, radiation therapy, phlebotomy, blood cell transfusion, bone marrow transplantation, peripheral stem cell transplantation, umbilical cord blood transplantation, autologous stem cell transplantation, allogeneic stem cell transplantation, syngeneic stem cell transplantation, surgery, induction therapy, maintenance therapy, and other therapy.

In some embodiments, cells (e.g. normal cells) other than the cells associated with a condition (e.g. cancer cells) or a combination of cells are used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. For example, in the case of cancer, infiltrating immune cells might determine the outcome of the disease. Alternatively, a combination of information from the cancer cells plus the immune cells in the blood that respond to the disease, or react to the disease can be used for diagnosis or prognosis of the cancer. Alternatively, in some embodiments, the invention is used to analyze cell samples, including, but not limited to cell lines, primary samples of solid or hematologic tissue, cultured cells, individual cell classes or sub-populations or mixtures thereof, and non-human cells.

In some embodiments, the analysis involves looking at multiple characteristics of the cell in parallel after contact with the compound. For example, the analysis can examine drug transporter function; drug transporter expression; drug metabolism; drug activation; cellular redox potential; signaling pathways; DNA damage repair; and apoptosis. Analysis can assess the ability of the cell to undergo cell cycle arrest and/or apoptosis after exposure to an experimental drug in an in vitro assay as well as the rate of drug export outside the cell or the rate of drug metabolism.

In some embodiments, the invention provides methods for classifying a cell population or determining the presence or absence of a condition in an individual by subjecting a cell from the individual to a modulator and/or a separate inhibitor, determining the activation level of an activatable element in the cell, and determining the presence or absence of a condition based on the activation level. In some embodiments, the activation level of a plurality of activatable elements in the cell is determined. The inhibitor can be an inhibitor as described herein. In some embodiments, the inhibitor is a phosphatase inhibitor. In some embodiments, the inhibitor is H₂O₂. The modulator can be any modulator described herein. In some embodiments, the methods of the invention provide for methods for classifying a cell population by exposing the cell population to a plurality of modulators in separate cultures and determining the status of an activatable element in the cell population. In some embodiments, the status of a plurality of activatable elements in the cell population is determined. In some embodiments, at least one of the modulators of the plurality of modulators is an inhibitor. The modulator can be at least one of the modulators described herein. In some embodiments, at least one modulator is selected from the group consisting of SDF-1α, IFN-α, IFN-γ, IL-10, IL-6, IL-27, G-CSF, FLT-3L, M-CSF, SCF, PMA, Thapsigargin, H₂O₂, etoposide, AraC, daunorubicin, staruosporine, and benzyloxycarbonyl-Val-Ala-Asp (OMe) fluoromethylketone (ZVAD-fmk), IL-3, IL-4, GM-CSF, EPO, LPS, TNF-α, CD40L, ON-01910.Na, cytidine analogs such as the Vidaza® cytidine analog, Dacogen® cytidine analog, paclitaxel, docetaxel, monastrol, doxorubicin, methotrexate, 5-fluorouricil, cisplatin, carboplatin, vincristine, bleomycin, flavopiridol, CY-202, maleic anhydride derivatives, BI2536, AZD5438, flavopiridol, roscovitine, R547, BMS-387032, UCN-01, K252a, olomucine II, fisetin, purvalanol A, isopentenyladenine, CVT-31351, bohemine, NU2058, AZ703, CGP-60474, PD0332991, indirubin, 7B10, E226, PHA-533533, STG28, Alsterpaullone, Kenpaullone, hymenialdisine, butyrolactone, GW9499, GW5181, acetophthalidin, methylselenocysteine, JNJ-7706621, BMI1026, and any combination thereof. The above listed modulators are useful, among other things, in hematopoietic cells for use in monitoring hematological disorders or as surrogate markers for non-hematological disorders (e.g. solid tumors). Other modulators can also be used such as EGF family ligands, PDGF family ligands, FGF family ligands, VEGF family ligands, Ang1, Ang2, HGF and IGF1. Some modulators can be a chemically synthesized inhibitor and some modulators can be a cellularly made inhibitor. In other embodiments, some modulators can be both a chemically synthesized and naturally made inhibitor, such as peroxide.

In some embodiments of the invention, the activation state of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the invention provides methods for determining a phenotypic profile of one or a population of cells by exposing the one or a population of cells to one or more of a plurality of modulators (recited herein) in separate cultures, wherein at least one of the modulators is an inhibitor, determining the presence or absence of an increase in the activation level of an activatable element in the cell population from each of the separate cultures and classifying the cell population based on the presence or absence of the increase in the activation level of the activatable element from each of the separate cultures. In some embodiments, the inhibitor is a cell cycle inhibitor, such as those described below.

Patterns and profiles of one or more activatable elements are detected using methods known in the art including those described herein. In some embodiments, patterns and profiles of activatable elements that are cellular components of a cellular pathway are detected using the methods described herein. For example, patterns and profiles of one or more phosphorylated polypeptides are detected using methods known in art including those described herein.

In some embodiments, the invention provides methods to carry out multiparameter flow cytometry for monitoring phospho-protein responses to various factors in myeloproliferative neoplasms at the single cell level. Phospho-protein members of signaling cascades and the kinases and phosphatases that interact with them are required to initiate and regulate proliferative signals in cells. Apart from the basal level of protein phosphorylation alone, the effect of potential drug molecules on these network pathways can be studied to discern unique cancer network profiles, which correlate with the genetics and disease outcome. Single cell measurements of phospho-protein responses reveal shifts in the signaling potential of a phospho-protein network, enabling categorization of cell network phenotypes by multidimensional molecular profiles of signaling. See U.S. Pat. No. 7,393,656. See also Irish et. al., Single cell profiling of potentiated phospho-protein networks in cancer cells. Cell. 2004, vol. 118, p. 1-20.

Flow cytometry is useful in a clinical setting, since relatively small sample sizes, as few as 10,000 cells, can produce a considerable amount of statistically tractable multidimensional signaling data and reveal key cell subsets that are responsible for a phenotype. See U.S. Pat. Nos. 7,381,535 and 7,393,656, and also Krutzik et al., 2004).

In some embodiments, the invention provides methods to determine dosing and scheduling of drugs. Drug selection, dosing, and dosing schedules can be guided by the effect of the drug on activatable elements in patient cells. In some embodiments, the invention may identify whether a patient responds to a drug, and therefore may be used to identify effective drugs for treating that patient. In some embodiments, the invention may be used to select drugs for combination therapies based on how a primary drug affects cell signaling or cell cycle progression in cell lines or patient samples: the invention may identify side effects, or biological processes that decrease efficacy of the drug. Based on these observations, combination treatments may be selected based on their ability to reduce side effects or enhance the efficacy of the primary drug. For example, the DNA methyltransferase inhibitors Vidaza® cytidine analog (5-Azacytidine) and Dacogen® cytidine analog (5-Aza-2′-deoxycytidine) are used to treat Acute Myeloid Leukemia (AML), a disease characterized by the overproliferation of undifferentiated cells. See U.S. Ser. No. 61/120,320, hereby incorporated by reference, for a more detailed description of AML, other hematologic malignancies, and current therapies and their mechanisms of action. Overexpression of DNA methyltransferases DNMT1, DMNT3a, and DMNT3b is associated with higher MDS disease risk. See Hopfer O. et al., Aberrant promoter methylation in MDS hematopoietic cells during in vitro lineage specific differentiation is differently associated with DNMT isoforms (2009), Leukemia Research 33 pp. 434-442; Langer, F. et al. (2005), Up-regulation of DNA methyltransferases DNMT1, 3A, and 3B in myelodysplastic syndrome, Leukemia Research 29, pp. 325-329, which are hereby incorporated by reference.

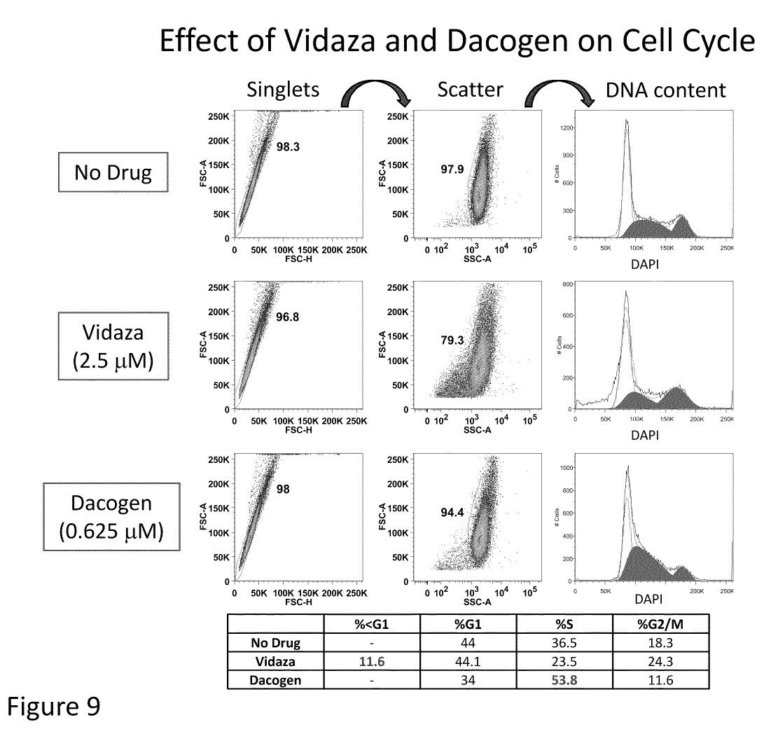

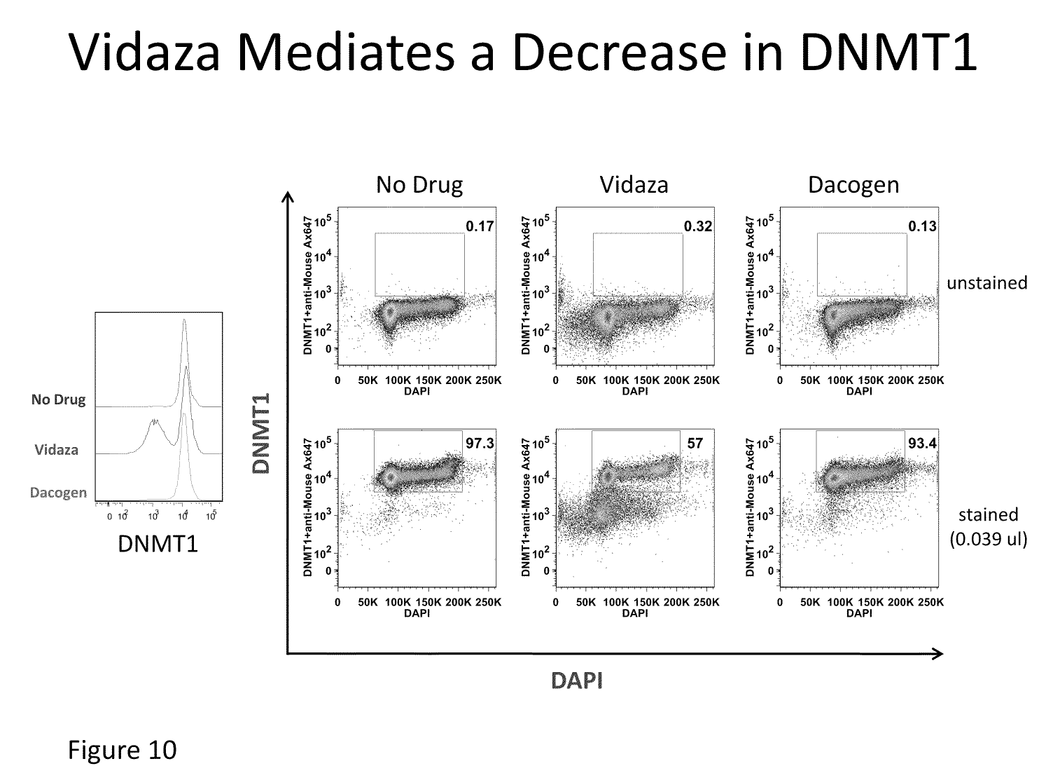

Vidaza® cytidine analog and Dacogen® cytidine analog are both pyrimidine analogs that inhibit DNA methyltransferase activity by incorporating into nucleic acids. By promoting DNA demethylation, Vidaza® cytidine analog and Dacogen® cytidine analog affect regulation of cells, such as cells affected by AML. Other drugs for the treatment of cancers, such as AML, include: Arsenic trioxide (apoptosis inducer), sorafenib (tyrosine kinase inhibitor), gemtuzumab ozogamicin (Mylotarg), vorinostat and valproic acid (histone deacetylase inhibitors), tipifarnib and lonafarnib (farnesyl transferase and RAF/RAS/ERK inhibitor), bevacizumab and ranibizumab (anti-EDGF monoclonal antibody that inhibits angiogenesis), ezatiostat (glutathione S1 transferase inhibitor), and clofarabine (nucleoside analog). A combination of hypomethylating agents with histone deacetylase (HDAC) inhibitors (MGCD-0103) is under trial for MDS and preliminary data suggests major responses (Itzykson et al., Meeting report: myelodysplastic syndromes at ASH 2007, Leukemia (2008) vol. 22 (5) pp. 893-7. See also Griffiths, E. A., and Gore, S. D., DNA Methyltransferase and Histone Deacetylase Inhibitors in the Treatment of Myelodysplastic Syndromes, Semin. Hematol. (2008) January 45(1) pp. 23-30. As one embodiment of the invention demonstrates, Vidaza® cytidine analog and Dacogen® Dacogen cytidine analog treatments elicit different responses as measured by different responses within different phases of the cell cycle, such as can be seen with Dacogen® cytidine analog inducing arrest at S phase, and Vidaza® cytidine analog inducing cell death (See Example 2; FIGS. 9-10).

The methods in this embodiment can be used to determine whether a patient responds to either Vidaza® cytidine analog, Dacogen® cytidine analog, or another drug that can be used to treat any cell cycle related disorder, such as AML, among other diseases. The methods in this embodiment can also be used to screen different combinations of drugs, such as a combination of hypomethylating agents and HDAC inhibitors. Additionally, the methods in this embodiment may be used to select a drug that induces entry into G2/M, cell cycle arrest, or apoptosis, as well as use in combination with Dacogen® cytidine analog or Vidaza® cytidine analog, or another drug that can be used to treat any cell cycle related disorder to increase overall treatment efficacy.

### Disease Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are well known in the art. In some embodiments the cellular pathway is a signaling pathway. Signaling pathways are also well-known in the art (see, e.g., Hunter T., Cell 100(1): 113-27 (2000); Cell Signaling Technology, Inc., 2002 Catalogue, Pathway Diagrams pgs. 232-253). It is also well-known in the art that disruptions of the cell cycle and/or inhibition of proapoptotic pathways, for example by genetic mutation or epigenetic modification, can cause or partially cause cancers and other disease states (for a detailed description, see Weinberg, The Biology of Cancer, 2007; Alberts, The Molecular Biology of the Cell, 4^{th} Ed., 2002; and Danial & Korsmeyer, Cell Death: Critical Control Points, 116 Cell 205 (2004)). A condition involving or characterized by altered physiological status may be readily identified, for example, by determining the state in a cell of one or more activatable elements, as taught herein. See U.S. Ser. No. 61/120,320.

In some embodiments, the present invention is directed to methods for analyzing the effects of a compound in one or more cells in a sample derived from an individual having or suspected of having a condition, which includes a cancer. For example, conditions include any solid or hematological cancer. Examples also include autoimmune, diabetes, cardiovascular, metabolic disorder, degenerative/wasting, neurological, endocrine, viral and other disease conditions. In some embodiments, the invention allows for identification of prognostically and therapeutically relevant subgroups of the conditions and prediction of the clinical course of an individual. Cell lines may also be used for testing.

One embodiment of the invention is a method to identify a proliferating cell or population of cells by measuring components of the cell cycle that indicate whether a cell is proliferating. Another embodiment of the invention is a method to identify in which phase of the cell cycle a cell is in by measuring components of the cell cycle that indicate which phase of the cell cycle the cell is in. This can be useful, for example, for selection of drug treatment, since some drugs exhibit greater efficacy on cells in a particular cell cycle phase. One embodiment of the invention is a method of determining when to administer a drug by identifying which phase of the cell cycle a cell is in. By knowing which phase a cell is in allows administration of a drug at a time when the drug can be more efficacious. For example, some drugs are more useful when administered during the G2/M phase of the cell cycle. A subject can have one or more cells analyzed to determine the cell cycle phase of the cell, for example, by determining the activation level of an activatable element using methods of the present invention, and then administered a drug when the cell is in a particular cell cycle phase. This can be a predetermined cell cycle phase. In some embodiments, the drug can be administered via a different dosing schedule or amount, based on the determination of the cell cycle phase. In some embodiments, the subject can be administered a first compound before analysis of the cell cycle phase, such as a compound that can arrest a cell in a particular cell cycle phase. In some embodiments, the methods can be used to ameliorate a disease or disorder, such as a cell cycle pathway disorder.

Another embodiment is a method for drug development in order to address “on” or “off” target activity of a drug. For example, a drug that is meant to bind to a particular target can be examined for binding to other pathway targets. Another embodiment of the invention is useful in patient selection in order to determine the likelihood of a patient responding based on the number of cycling cells in a specimen. Another embodiment of the invention is useful in patient selection in order to determine the likelihood of a patient responding based on particular pathway activation. Specimens may include bone marrow, peripheral blood, biopsy fine needle aspirates, circulating tumor cells, and the like. Another embodiment of the invention is a method for detecting a combination of therapeutic agents that include inhibiting cell proliferation.

One embodiment of the invention provides methods to characterize cell cycle and cell death pathway alterations found in disease conditions such as any solid or hematological cancer, immune, autoimmune, diabetes, cardiovascular, metabolic disorder, degenerative/wasting, neurological, endocrine, viral and other disease conditions, such as Myelodysplastic Syndromes (MDS). MDS may be caused by chromosome eight trisomy and is characterized by bone marrow failure and increased survival of immature myeloid cells called blasts. Increased blast survival may result from both cell cycle and apoptotic defects. MDS blasts may display increased proliferative capacity due to cell cycle dysregulation and may also display increased cell survival due to a diminished ability of blasts to respond to proapoptotic signals. Accordingly, one symptom of MDS is an expansion of CD34+ blasts. This expansion may be caused by upregulation of the antiapoptotic proteins survivin and c-myc coupled with increased expression of cell cycle regulators such as Cyclin D. Thus, one embodiment of the invention measures regulation of survivin, c-myc, and/or cyclin D for characterization of cell cycle and cell death pathway alterations in a disorder, such as MDS.

One embodiment of the invention may evaluate the efficacy of a compound designed to target a cell cycle regulator, such as ON-01910.Na. For example, evaluation of ON-01910.Na can be done in the TF-1 cell line, an in vitro model of MDS. Because ON-01910.Na targets cell cycle regulators including, but not limited to Polo-like kinase (Plk1) and cyclin dependent kinase 1 (Cdk1), cell cycle and apoptotic pathways may be monitored alone or together as described below.

Another embodiment of the invention provides methods to monitor the effect of a compound on cell cycle progression and determine the cell cycle phase of a single cell or a population of cells. The cell cycle phase of proliferating, cycling cells may be determined by monitoring the activation level of activatable elements within Cyclin B1, Cdk1, Cdc25, Plk1, and Histone H3. A determination of total DNA content within a single cell or population of cells may also be used to determine the cell cycle phase of a single cell or a population of cells.

In some of these embodiments, polypeptides may be used to monitor cell cycle progression because the activation levels of their various activatable elements change during cell cycle progression. In particular, Cyclin B1 expression levels increase during G2 and remain high through M phase, phosphorylation of the Cyclin B1/Cdk1 complex at tyrosine 15 (Y15) decreases as cells transition from G2 to M phase while phosphorylation of threonine 161 (T161) increases as cells enter M phase, and histone 3(H3) phosphorylation at serine 28 (S28) increases as cells transition into M phase. Plk1 becomes activated by phosphorylation at serine 137 (S137) and threonine 210 (T210) during G2 and remains activated through M phase. Active Plk1 then activates Cdc25 by directly phosphorylating serine 198 (S198). See L. Tsvetkov & D. F. Stern, Phosphorylation of Plk1 at S137 and T210 is inhibited in response to DNA damage, 4 Cell Cycle 166 (2005). The phosphorylation state of at least one of these residues or any combination thereof may be monitored as described below to determine the cell cycle phase of a single cell or a population of cells.

The DNA content of a single cell or a population of cells may be monitored to simultaneously determine both the cell cycle phase and cell death status of the single cell or population of cells. Cellular DNA content reveals the cell cycle phase of a cell because the cellular genome is duplicated once per cell cycle. Somatic cells will generally have pairs of chromosomes; for example, humans have 23 pairs of chromosomes. This level of DNA content is termed 2n in the art where n denotes a number of chromosomes that is characteristic of different species. As cells progress through the cell cycle, the genome is duplicated during S phase and at the conclusion of a normal S phase, a cell will have doubled the pairs of all chromosomes or have 4n DNA content. Quiescent, or nonproliferating cells and cells in G1 phase typically have 2n DNA content, while cells in the G2 and M phases will have 4n DNA content and S phase cells have an intermediate level of DNA as genome replication is not yet complete.

DNA content may also be used to monitor death of a single cell or the amount of death within a population of cells. Cellular DNA is degraded or cleaved between histones during nonapoptotic and apoptotic cell death respectively. Such genomic degradation ultimately eliminates a significant proportion of cellular DNA such that 2n or 4n DNA content is reduced to sub-2n levels in dead or dying cells. Cells having sub-2n levels are indicative of dead or dying cells, such as cells undergoing apoptosis. The amount of sub-2n (also termed sub-G1) DNA content is proportional to the amount of cell death within a sample.

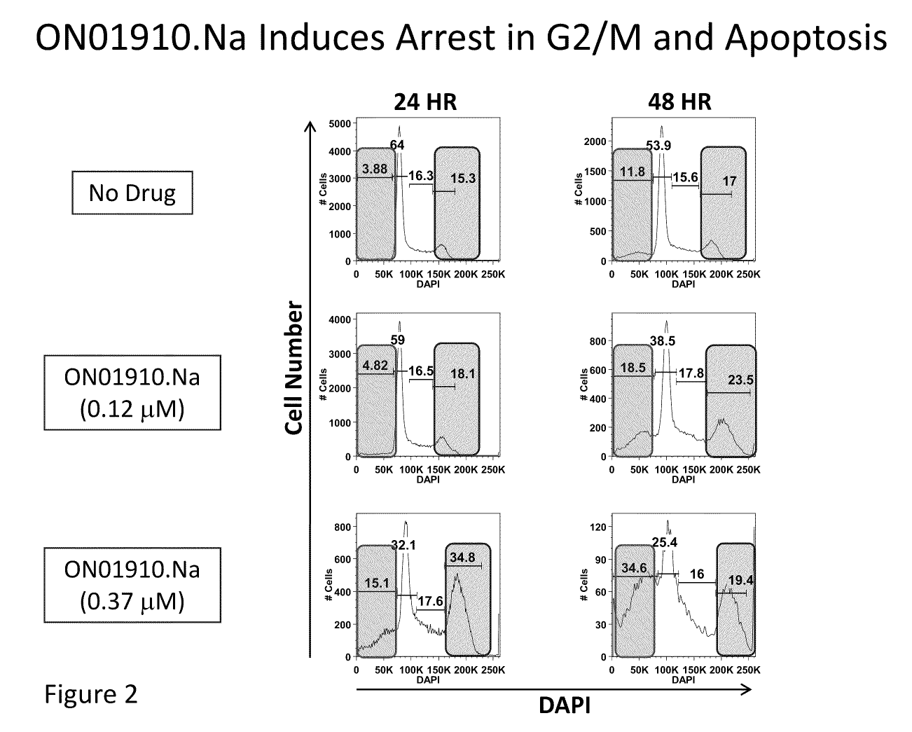

DNA content can be directly monitored using fluorescent dyes that bind DNA in the major or minor groove. This labeled DNA can be detected and cellular DNA content can be determined using flow cytometry or other methods as described below. DNA content can be used to indicate the status of a cell or population of cells. The effect of a compound on both the cell cycle and cell death pathways can be determined by monitoring changes in DNA content within a single cell or a population of cells. For example, cell cycle arrest in G1 or G2/M phases of the cell cycle may appear as an increase in 2n or 4n DNA content respectively while an increase in cell death may appear as an increase in sub-2n DNA content. For example, FIG. 2 demonstrates that the compound ON-01910.Na induces arrest in G2/M and cell death within treated TF-1 cells. The 4n DNA content peak increases in a dose-dependent manner when cells are treated with 0.12 μM and 0.37 μM ON-01910.Na indicating that this compound causes cell cycle arrest and cell death.

Yet another embodiment of the invention provides methods to assess the extent of cell death or apoptosis in a single cell or population of cells. In particular, the invention provides methods to determine cell death after treatment of a sample with a compound. Apoptosis is a form of cell death regulated by cellular pathways, and the invention provides methods to determine the activation level of at least one activatable element that regulates apoptosis.

Apoptotic regulators, or nodes, that can be monitored include, but are not limited to caspase-8 to determine activation of the extrinsic, receptor mediated pathway, cytochrome c release from the intermembrane space of the mitochondria to determine activation of the intrinsic pathway, upregulation of Bcl-2 and survivin expression to determine dysregulation of pro-survival pathways, caspase-3 and PARP to determine late apoptotic events proximal to engulfment, and Chk2 and histone 2AX (H2AX) phosphorylation to determine any crosstalk between activation of the DNA damage response and apoptosis pathways. Other apoptotic related proteins that can be monitored, include, but are not limited to TNF receptors, TRADD, FADD, Fas receptor, FasL, caspase-10, BAX, BID, BAK, BAD, Bcl-X_{L}, SMAC, VDAC2, and AIF. Any of the nodes listed above may be monitored in the presence or absence of a modulator, and any node may be monitored alone or in any combination.

### Compounds to be Analyzed

Compounds analyzed in some embodiments of the present invention can be designed to treat cancer, autoimmune and other diseases. In some embodiments, the compounds can induce cell death, apoptosis or halt disease progression. In some embodiments, the compounds can affect cell cycle components and regulators. In some embodiments, the compounds can inhibit DNA methylation. See also U.S. Ser. No. 61/120,320, hereby incorporated by reference, for a description of compounds that affect DNA methylation. In some embodiments the compounds can damage DNA. In some embodiments the compounds can induce apoptosis or nonapoptotic cell death. Active compounds include agents that can target the cell cycle and can induce cell death or apoptosis. These agents can be common cytotoxic agents that are used in cancer chemotherapy, or any other agents that are generally cytostatic or cytotoxic.

### Activatable Elements

The methods and compositions of the invention may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously). The cell can be a hematopoietic cell or one which originates from a solid tumor.

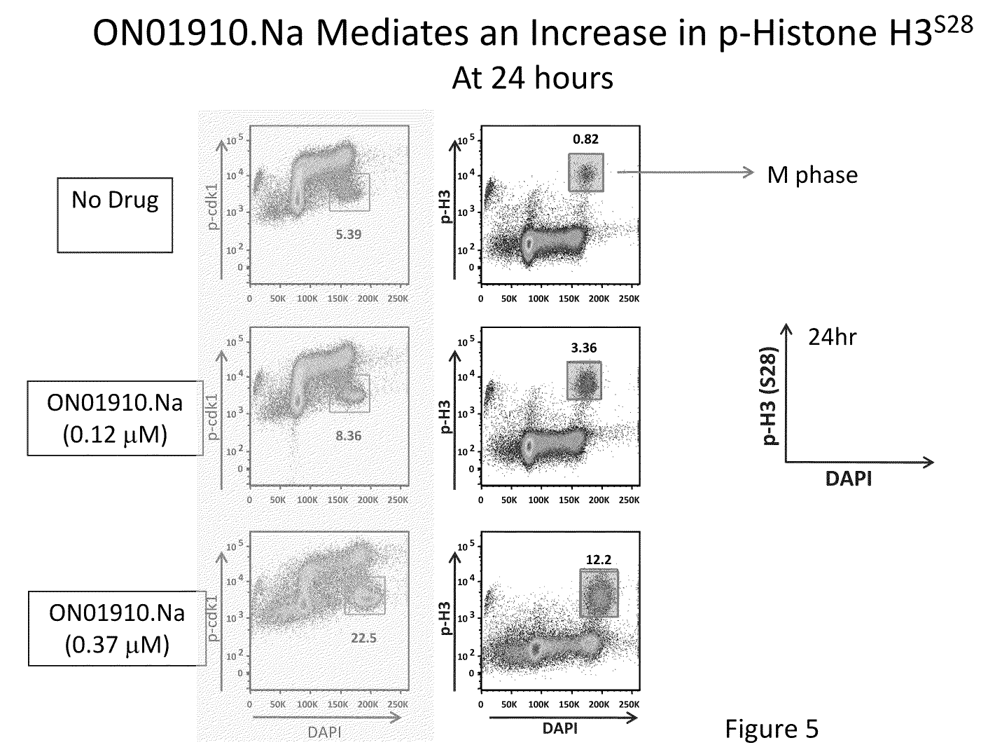

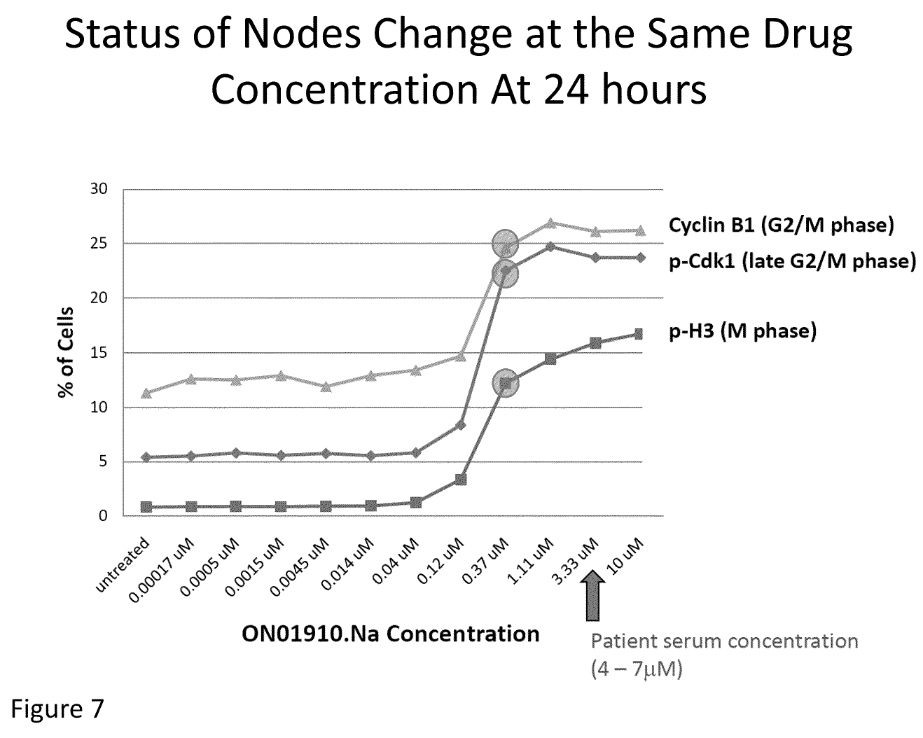

One method of the invention determines levels of activation of components within the cell cycle in single cells. See FIGS. 5, 6, 9, and 10. One method of the invention determines how levels of activation of components within the cell cycle are affected by levels and activation states of pro- and anti-apoptotic molecules. See FIG. 7. One method determines how progression through the cell cycle is affected by treating cells with compounds that reduce DNA methylation. See FIG. 35.

Examples of hematopoietic cells include, but are not limited to pluripotent hematopoietic stem cells, granulocyte lineage progenitor or derived cells, monocyte lineage progenitor or derived cells, macrophage lineage progenitor or derived cells, megakaryocyte lineage progenitor or derived cells and erythroid lineage progenitor or derived cells. As a non-limiting example, the cells may also come from solid tumors as circulating tumor cells, ascites from ovarian cancer, and cells derived from larger masses, such as from biopsies. Circulating tumor cells may be rare cells, see U.S. Ser. No. 61/048,886.

As will be appreciated by those in the art, a wide variety of activation events can find use in the present invention. In general, the basic requirement is that the activation results in a change in the activatable element that is detectable by some indication (termed an “activation state indicator”), preferably by altered binding of a labeled binding element or by changes in detectable biological activities (e.g., the activated state has an enzymatic activity which can be measured and compared to a lack of activity in the non-activated state). What is important is to differentiate, using detectable events or moieties, between two or more activation states.

As an illustrative example, and without intending to be limited to any theory, an individual phosphorylatable site on a protein can activate or deactivate the protein. Additionally, phosphorylation of an adapter protein may promote its interaction with other components/proteins of distinct cellular signaling pathways. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may or may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes of cells that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a cell is classified according to the activation levels of a plurality of activatable elements. In some embodiments, 1, 2, 3, 4, 5, 6, 7, 8, 9, 10 or more activatable elements may be analyzed in a cell signaling pathway. In some embodiments, the activation levels of one or more activatable elements of a cell are correlated with a condition. In some embodiments, the activation levels of one or more activatable elements of a cell are correlated with a neoplastic condition as described herein.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by interacting with other biomolecules, by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. Activation states of activatable elements may result from chemical additions or modifications of biomolecules and include many biochemical processes. See U.S. Application No. 61/085,789, which is incorporated by reference.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell or to measure the activation state of activatable elements, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, cell cycle progress can be inferred by measuring levels of cyclin proteins.

In alternative embodiment, activation of the activatable element is detected as intermolecular clustering of the activatable element. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

Clusters can be made up of identical elements or different elements. Clusters of identical elements are termed “homo” dimers, while clusters of different elements are termed “hetero” clusters. Accordingly, a cluster can be a homodimer, as is the case for the β₂-adrenergic receptor.

Alternatively, a cluster can be a heterodimer, as is the case for GABA_{B-R}. In other embodiments, the cluster is a homotrimer, as in the case of TNFα, or a heterotrimer such the one formed by membrane-bound and soluble CD95 to modulate apoptosis. In further embodiments the cluster is a homo-oligomer, as in the case of Thyrotropin releasing hormone receptor, or a hetero-oligomer, as in the case of TGFβ1. One embodiment includes hetero and homo dimmers of the EGF receptor (HER) family of receptor tyrosine kinases.

In a preferred embodiment, the activation or signaling potential of elements is mediated by clustering, irrespective of the actual mechanism by which the element's clustering is induced. For example, elements can be activated to cluster a) as membrane bound receptors by binding to ligands (ligands including both naturally occurring or synthetic ligands), b) as membrane bound receptors by binding to other surface molecules, or c) as intracellular (non-membrane bound) receptors binding to ligands.

In a preferred embodiment the activatable elements are membrane bound receptor elements that cluster upon ligand binding such as cell surface receptors. As used herein, “cell surface receptor” refers to molecules that occur on the surface of cells, interact with the extracellular environment, and transmit or transduce (through signals) the information regarding the environment intracellularly in a manner that may modulate cellular activity directly or indirectly, e.g., via intracellular second messenger activities or transcription of specific promoters, resulting in transcription of specific genes. One class of receptor elements includes membrane bound proteins, or complexes of proteins, which are activated to cluster upon ligand binding. As is known in the art, these receptor elements can have a variety of forms, but in general they comprise at least three domains. First, these receptors have a ligand-binding domain, which can be oriented either extracellularly or intracellularly, usually the former. Second, these receptors have a membrane-binding domain (usually a transmembrane domain), which can take the form of a seven pass transmembrane domain (discussed below in connection with G-protein-coupled receptors) or a lipid modification, such as myristylation, to one of the receptor's amino acids which allows for membrane association when the lipid inserts itself into the lipid bilayer. Finally, the receptor has a signaling domain, which is responsible for propagating the downstream effects of the receptor.

Examples of such receptor elements include hormone receptors, steroid receptors, cytokine receptors, such as IL1-α, IL-β, IL-2, IL-3, IL-4, IL-5, IL-6, IL-7, IL-8, IL-9, IL-10. IL-12, IL-15, IL-18, IL-21, CCR5, CCR7, CCR-1-10, CCL20, chemokine receptors, such as CXCR4, adhesion receptors and growth factor receptors, including, but not limited to, PDGF-R (platelet derived growth factor receptor), EGF-R (epidermal growth factor receptor), VEGF-R (vascular endothelial growth factor), uPAR (urokinase plasminogen activator receptor), ACHR (acetylcholine receptor), IgE-R (immunoglobulin E receptor), estrogen receptor, thyroid hormone receptor, integrin receptors (β1, β2, β3, β4, β5, β6, α1, α2, α3, α4, α6, α6), MAC-1 (β2 and cd11b), αVβ33, opioid receptors (mu and kappa), FC receptors, serotonin receptors (5-HT, 5-HT6, 5-HT7), β-adrenergic receptors, insulin receptor, leptin receptor, TNF receptor (tissue-necrosis factor), statin receptors, FAS receptor, BAFF receptor, FLT3 LIGAND receptor, GMCSF receptor, and fibronectin receptor.

The receptor tyrosine kinases can be divided into subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the EPH/ECK family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence (Hanks et al., (1988) Science 241:42-52). Subgroups III (platelet-derived growth factor (PDGF) receptor-like) and IV (the fibro-blast growth factor (FGF) receptors) are characterized as having immunoglobulin (Ig)-like folds in their extracellular domains, as well as having their kinase domains divided in two parts by a variable stretch of unrelated amino acids (Yarden and Ullrich (1988) supra and Hanks et al., (1988) supra). For further discussion, see U.S. Patent Application 61/120,320.

In a further embodiment, the receptor element is an integrin other than Leukocyte Function

Antigen-1 (LFA-1). Members of the integrin family of receptors function as heterodimers, composed of various a and subunits, and mediate interactions between a cell's cytoskeleton and the extracellular matrix. (Reviewed in Giancotti and Ruoslahti, Science 285, 13 Aug. 1999). Different combinations of the α and β subunits give rise to a wide range of ligand specificities, which may be increased further by the presence of cell-type-specific factors. Integrin clustering is known to activate a number of intracellular pathways, such as the RAS, Rab, MAP kinase pathway, and the PI3 kinase pathway. In a preferred embodiment the receptor element is a heterodimer (other than LFA-1) composed of a integrin and an α integrin chosen from the following integrins; β1, β2, β3, β4, β5, β6, α1, α2, α3, α4, α5, and α6, or is MAC-1 (β2 and cd11b), or αVβ3.

In a preferred embodiment the element is an intracellular adhesion molecule (ICAM). ICAMs-1, -2, and -3 are cellular adhesion molecules belonging to the immunogloblin superfamily. Each of these receptors has a single membrane-spanning domain and all bind to β2 integrins via extracellular binding domains similar in structure to Ig-loops. (Signal Transduction, Gomperts, et al., eds, Academic Press Publishers, 2002, Chapter 14, pp 318-319).

In another embodiment the activatable elements cluster for signaling by contact with other surface molecules. In contrast to the receptors discussed above, these elements cluster for signaling by contact with other surface molecules, and generally use molecules presented on the surface of a second cell as ligands. Receptors of this class are important in cell-cell interactions, such mediating cell-to-cell adhesion and immunorecognition.

Examples of such receptor elements are CD3 (T cell receptor complex), BCR (B cell receptor complex), CD4, CD28, CD80, CD86, CD54, CD102, CD50 and ICAMs 1, 2 and 3.

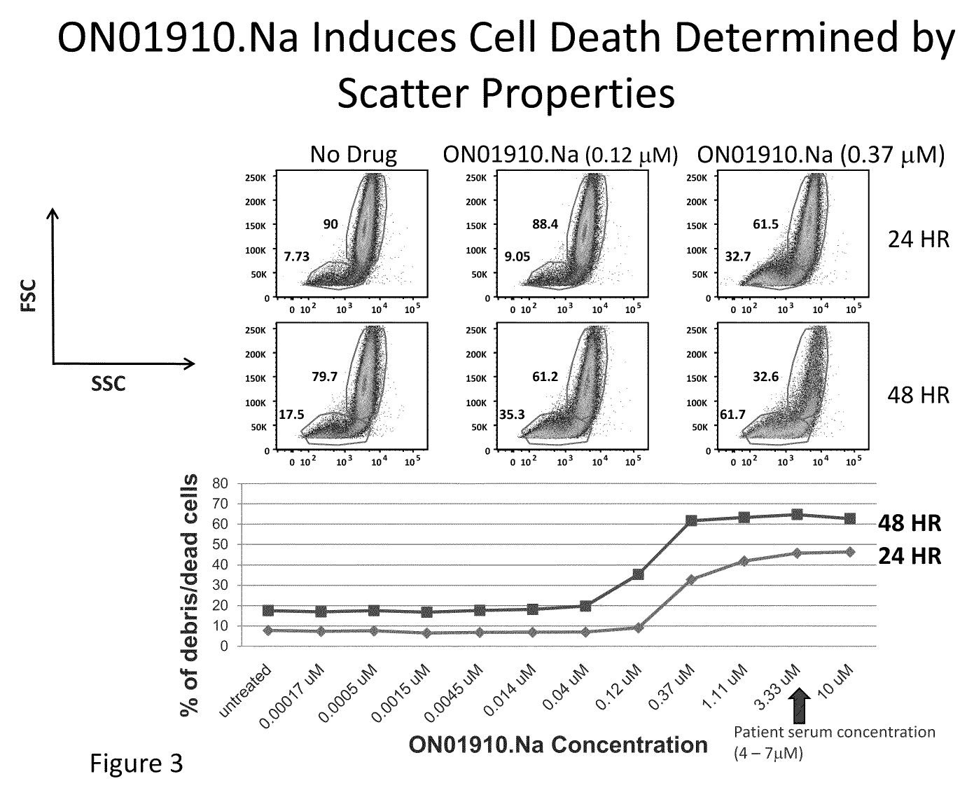

In some embodiments of the invention, the activatable elements may function in cell death, including apoptosis or necrosis. A person of ordinary skill in the art may analyze cell death using stains, biomarkers, assays, or kits to identify node states associated with cell cycle progression and cell death without departing from the scope of the invention. By way of example, stains used to identify cell death include, but are not limited to amine aqua, propidium iodide, 4′,6-diamidino-2-phenylindole (DAPI), bromodeoxyuridine (BrdU), acridine orange, SYTOX, and TUNEL. A person of ordinary skill in the art will appreciate that several of the aforementioned stains, such as DAPI, may also be used to determine the cell cycle of a single cell or population of cells. Cell death may also be identified using the forward versus side scatter dot plots obtained during flow cytometry. FIG. 3 illustrates that the compound ON-01910.Na induces cell death as determined by the scatter properties of the treated cells.

In one embodiment, the activatable elements are intracellular receptors capable of clustering. Elements of this class are not membrane-bound. Instead, they are free to diffuse through the intracellular matrix where they bind soluble ligands prior to clustering and signal transduction. In contrast to the previously described elements, many members of this class are capable of binding DNA after clustering to directly effect changes in RNA transcription.

In another embodiment the activatable element is a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalytic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Chim C. S., Wong K Y, Loong F, Srivastava G., SOCS1 and SHP1 hypermethylation in mantle cell lymphoma and follicular lymphoma: implications for epigenetic activation of the Jak/STAT pathway, Leukemia, February 2004, 18(2): 356-8.

In another embodiment, the activatable element is a microRNA. MicroRNAs (miRNAs) are non-coding RNA molecules, approximately 22 nucleotides in length, which play important regulatory roles in gene expression in animals and plants. mRNAs modulate gene flow through post-transcriptional gene silencing through the RNA interference pathway. Once one strand of miRNA is incorporated into the RNA induced silencing complex (RISC), it interacts with the 3′ untranslated regions (UTRs) of target mRNAs through partial sequence complementarity to bring about translational repression or mRNA degradation. The net effect is to downregulate the expression of the target gene by preventing the protein product from being produced. Mirnezami et al., MicroRNAs: Key players in carcinogenesis and novel therapeutic agents, Eur. J. Surg. Oncol., Jun. 9, 2006, doi:10.1016/j.ejso.2008.06.006, hereby fully incorporated by reference in its entirety.

The discovery of a novel class of gene regulators, named microRNAs (miRNAs), has changed the landscape of human genetics. miRNAs are ˜22 nucleotide non-coding RNA that regulate gene expression by binding to 3′ untranslated regions of mRNA. If there is perfect complementarity, the mRNA is cleaved and degraded whereas translational silencing is the main mechanism when base pairing is imperfect. Recent work has led to an increased understanding of the role of miRNAs in hematopoietic differentiation and leukemogenesis. Using animal models engineered to overexpress miR-150, miR-17 approximately 92 and miR-155 or to be deficient for miR-223, miR-155 and miR-17 approximately 92 expression, several groups have now shown that miRNAs are critical for B-lymphocyte development (miR-150 and miR-17 approximately 92), granulopoiesis (miR-223), immune function (miR-155) and B-lymphoproliferative disorders (miR-155 and miR-17 approximately 92). Distinctive miRNA signatures have been described in association with cytogenetics and outcome in acute myeloid leukemia. There is now strong evidence that miRNAs modulate not only hematopoietic differentiation and proliferation but also activity of hematopoietic cells, in particular those related to immune function. Extensive miRNA deregulation has been observed in leukemias and lymphomas and mechanistic studies support a role for miRNAs in the pathogenesis of these disorders (Garzon et al., MicroRNAs in normal and malignant hematopoiesis, Current Opinion Hematology, 2008, 15:352-8). miRNAs regulate critical cellular processes such as cell cycle, apoptosis and differentiation. Consequently impairments in their regulation of these functions through changes in miRNA expression can lead to tumorigenesis. miRNAs can act as oncogenes or tumor suppressors. miRNA profiles can provide important prognostic information as recently shown for acute myeloid leukemia (Marcucci et al., J. Clinical Oncology (2008) 26:p5078). In another study, Cimmino et al., (PNAS (2005) 102:p. 13944) showed that patients with chronic lymphocytic leukemia (CLL) have deletions or down regulation of two clustered miRNA genes; mir-15a and mir-16-1. These miRNAs negatively regulate the anti-apoptotic protein Bcl-2 that is often overexpressed in multiple malignancies including but not limited to leukemias and lymphomas. Thus, miRNAs are a potentially useful diagnostic tool in diagnosing cancer, classifying different types of tumors, and determining clinical outcome, including but not limited to, MPNs. A. Esquela-Kerscher and F. J. Slack, Oncomirs—microRNAs with a role in cancer, Nat. Rev. Cancer, April 2006, 6: 259-269 is hereby fully incorporated by reference.

In another embodiment the activatable element is a small molecule, carbohydrate, lipid or other naturally occurring or synthetic compound capable of having an activated isoform. In addition, as pointed out above, activation of these elements need not include switching from one form to another, but can be detected as the presence or absence of the compound. For example, activation of cAMP (cyclic adenosine mono-phosphate) can be detected as the presence of cAMP rather than the conversion from non-cyclic AMP to cyclic AMP.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886; 61/048,920; and Shulz et al., Current Protocols in Immunology 2007, 78:8.17.1-20.

In some embodiments, the protein with a potential activatable element is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon β, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, A1, Bax, Bak, Bok, Bik, Bad, Bid, Bim, Bmf, Hrk, Noxa, Puma, IAPB, XIAP, Smac, survivin, Plk1, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoA Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, DMNT1, DMNT3a, DMNT3b, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β̅-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

Generally, the methods of the invention involve determining the activation levels of an activatable element in a plurality of single cells in a sample. The activation levels can be obtained by perturbing the cell state using a modulator.

### Modulators

In some embodiments, the methods and composition utilize a modulator. A modulator can be an activator, a therapeutic compound, an inhibitor or a compound capable of impacting a cellular pathway or causing an effect in an activatable element, or some combination of the above. Modulators can also take the form of a variety of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See the U.S. patent applications recited above which are incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, candidate drugs molecules or compounds, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrates, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the invention can be used to evaluate at least 1, 2, 3, 4, 5, 6, 7, 8, 9, 10, 11, 12, or more dilutions of a modulator or combination of modulators at 1, 2, 3, 4, 5, 6, 7, 8, 9, 10 11, 12 or more timepoints. These dilutions series may be used to titrate the modulator in cell lines or patient samples in order to select a dosing and scheduling regimen. In some embodiments, the dilution series may be selected from a range: The range may have a minimum as low as no molecule, or 1×10⁻⁴ μM, or 1×10⁻³ μM, or 1×10⁻² μM and a maximum as high as 1×10⁻² μM, 1×10⁻¹ μM, 1 μM, or greater. Additionally, in some embodiments, the invention can be used to treat cells for durations of less than one minute, or for at least 1, 2, 3, 4, 5, 6, 7, 8, 9, 10, and up to 60 or more minutes and fractions thereof, or for 1, 2, 3, 4, 5, 6, 7, 8, 9, 10 or more hours and up to 24 hours and fractions thereof, or for 1, 2, 3, 4, 5, 6, 7, 8, 9, 10 or more days and fractions thereof. See FIGS. 3, 7, and 13 for examples of how some embodiments of the invention can be used in titration experiments that can be used for determining dosing and scheduling of a drug.

In some embodiments, the cross-linker is a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor.

In some embodiments, the activation level of an activatable element in a cell is determined by contacting the cell with an inhibitor and a separate modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In one embodiment the modulators affect apoptosis and the cell cycle. In another embodiment, the modulators are TNFα, FasL, G-CSF, IFN-α, β, and δ, Flt3L, SCF or anti-IgM antibody or fragment thereof. In yet another embodiment the modulators are selected from a group consisting of ON-01910.Na, Vidaza® cytidine analog, Dacogen® cytidine analog, paclitaxel, docetaxel, monastrol, doxorubicin, methotrexate, 5-fluorouracil, cisplatin, carboplatin, vincristine, bleomycin, flavopiridol, CY-202, maleic anhydride derivatives, BI2536, AZD5438, flavopiridol, roscovitine, R547, BMS-387032, UCN-01, K252a, olomucine II, fisetin, purvalanol A, isopentenyladenine, CVT-31351, bohemine, NU2058, AZ703, CGP-60474, PD0332991, indirubin, 7BIO, E226, PHA-533533, STG28, Alsterpaullone, Kenpaullone, hymenialdisine, butyrolactone, GW9499, GW5181, acetophthalidin, methylselenocysteine, JNJ-7706621, BMI1026, and any combination thereof.

In some embodiments, the modulator can be a targeted cell cycle modulator. A targeted cell cycle modulator has a direct effect on one or more components of the cell cycle pathway. For example, inhibitors that bind to a cyclin or cdk protein can have a direct effect on one or more components of the cell cycle pathway. As another example, direct inhibitors of DNA or RNA, such as nucleotide or nucleoside analogs can have a direct effect on one or more components of the cell cycle pathway. In some embodiments, the modulator can be a DNA methyltransferase, a DNA alkylating agent or a DNA methylating agent. In some embodiments, the modulator can be a growth factor inhibitor.

In some embodiments, the modulator can be a targeted cell cycle modulator that is a product that causes DNA damage, such as a natural product that causes DNA damage. Examples of products that causes DNA damage include, but are not limited to, bleomycin, daunorubicin, docetaxel, doxorubicin, epirubicin, etoposide, homoharringtonine, idarubicin, irinotecan, mitomycin, mitoxantrone, paclitaxel, topotecan, vinblastine, vincristine, or vinorelbine. In some embodiments, the modulator can be a targeted cell cycle modulator that is an alkylating agent. Examples of alkylating agents include, but are not limited to, altretamine, busulfan, carboplatin, chlorambucil, cisplatin, cyclophosphamide, dacarbazine, ifosfamide, lomustine, mechlorethamine, melphelan, or procarbazine.

In some embodiments, the modulator can be a targeted cell cycle modulator that is an antimetabolite. Examples of antimetabolites include, but are not limited to, azacytidine (nucleoside analog), cladribine (nucleoside analog), cytarabine (nucleoside analog), floxuridine (nucleoside analog), fludarabine (nucleoside analog), fluorouracil (nucleoside analog), edatrexate, gemcitabine (nucleoside analog), hydroxyurea, mercaptopurine, methotrexate, pentostatin, thioguanine (nucleoside analog) or tomudex (ZD 1694) (thymidylate synthase inhibitor).

### Gating

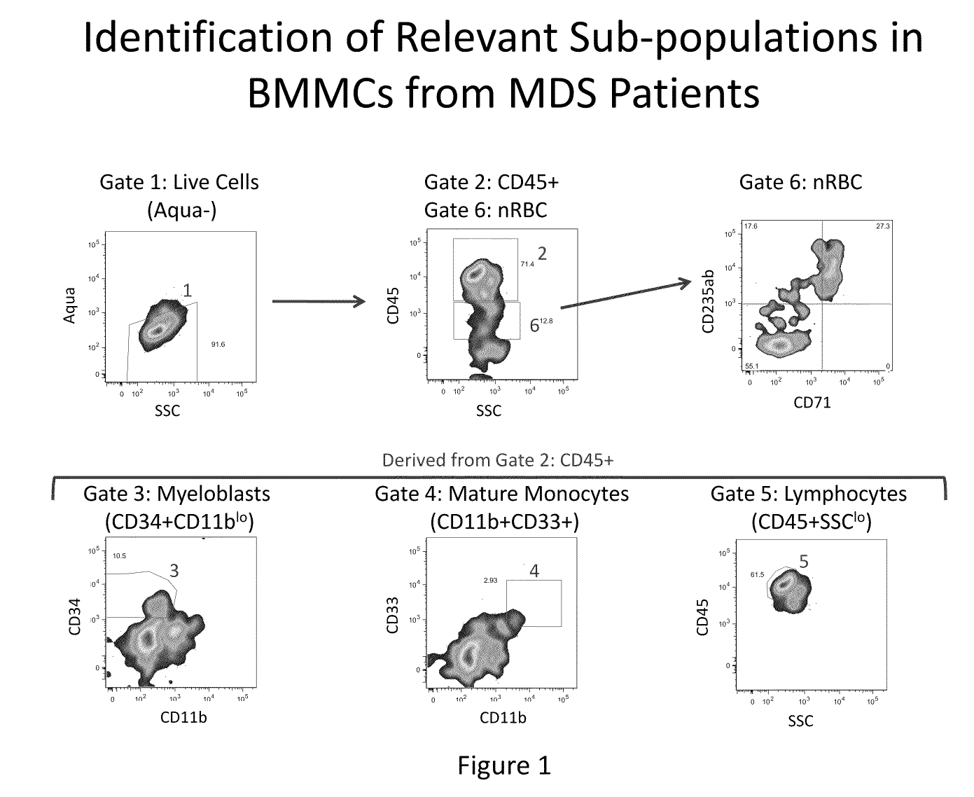

In some embodiments of the invention, different gating strategies can be used in order to analyze only relevant subpopulations of cells derived from a sample of mixed population. These gating strategies can be based on the presence of one or more specific surface marker expressed on each cell type. More than one gate may be applied to the sample of mixed population or a subpopulation. FIG. 1 shows an example gating strategy that identifies relevant subpopulations in BMMC samples taken from MDS patients. See U.S. Patent Applications 61/085,789, 61/120,320, and 61/079,766, hereby incorporated by reference.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See the above patents and applications for example methods.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

Detection of cell signaling states may be accomplished using binding elements and labels.

Cell signaling states may be detected by a variety of methods known in the art. They generally involve a binding element, such as an antibody, and a label, such as a fluorchrome to form a detection element. Detection elements do not need to have both of the above agents, but can be one unit that possesses both qualities. These and other methods are well described in U.S. Pat. Nos. 7,381,535 and 7,393,656 and U.S. Ser. Nos. 10/193,462; 11/655,785; 11/655,789; 11/655,821; 11/338,957, 61/048,886; 61/048,920; and 61/048,657 which are all incorporated by reference in their entireties.

In one embodiment of the invention, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions of the present invention, it will recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361.

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, one or more cells are contained in a well of a 96 well plate or other commercially available multiwell plate. In an alternate embodiment, the reaction mixture or cells are in a cytometric measurement device. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al., Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, DNA microarrays are commercially available through a variety of sources (Affymetrix, Santa Clara, Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, the methods of the invention include the use of liquid handling components. The liquid handling systems can include robotic systems comprising any number of components. In addition, any or all of the steps outlined herein may be automated; thus, for example, the systems may be completely or partially automated. See U.S. Patent Application Nos. 61/048,657. and 61/181,211.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradeable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology, February 2008. Methods for the analysis of multiple parameters are well known in the art. See U.S. Patent Application No. 61/079,579 or 12/501,295 for gating analysis. See U.S. patent application Ser. No. 12/460,029 for methods of analysis.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g. tumor cells). See also U.S. Patent Application No. 61/079,537 or 12/501,295 for visualization or gating tools.

### Kits

In some embodiments the invention provides kits. Kits provided by the invention may comprise one or more of the state-specific binding elements described herein, such as phospho-specific antibodies. A kit may also include other reagents that are useful in the invention, such as modulators, fixatives, containers, plates, buffers, stains and labeling reagents therapeutic agents, instructions, and the like.

In some embodiments, the kit comprises one or more antibodies that recognize non-phospho and phospho epitopes within a protein, including, but not limited to Lnk, SOCS3, SH2-B, Mpl, Epo receptor, and Flt-3 receptor. Another embodiment includes one or more antibodies that recognize non-phospho and phospho epitopes within a protein, including, but not limited to those shown in FIG. 9, such as the activatable elements for the cell cycle profile and apoptosis. Kits may also include instructions for use and software to plan, track experiments, and files which contain information to help run experiments.

Kits provided by the invention may comprise one or more of the modulators described herein.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer. Additionally, in some embodiments, kits may be marketed for drug screening applications

Examples that may serve to more fully describe the manner of using the above-described invention, as well as to set forth the best modes contemplated for carrying out various aspects of the invention can be seen in the incorporated application 61/120,320. It is understood that these examples in no way serve to limit the true scope of this invention, but rather are presented for illustrative purposes. All references cited herein are expressly incorporated by reference in their entireties

### Example 1

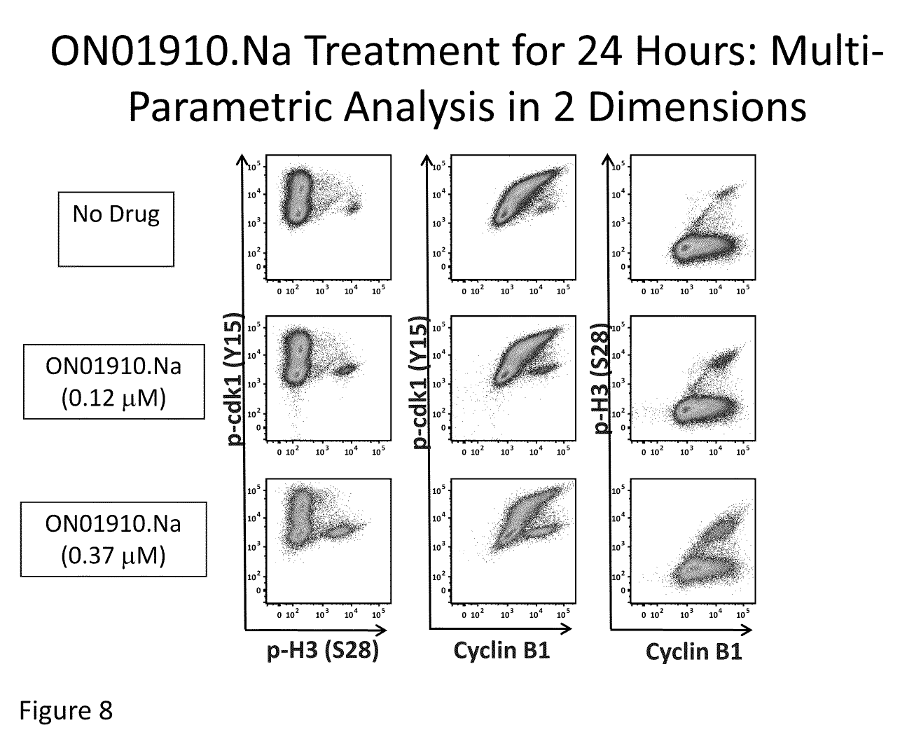

FIGS. 1 through 8 and 10 through 13 show an example of one embodiment of the present invention. General conditions, reagents, times, procedures were followed in a manner similar to those shown in the references cited above. See also U.S. Ser. No. 61/120,320. All of these references are hereby incorporated by reference.

In the example, erythroblast (TF-1) and U937 cell lines, as well as healthy bone marrow mononuclear cells, were treated with a test modulator (ON01910.Na) over several dilutions for 24 hours. Flow cytometry was used to obtain multiple intracellular readouts or nodes, including levels of protein phosphorylation, levels of protein expression, cell size and shape, and DNA content.

FIG. 2 shows that in erythroblasts ON01910.Na induces arrest in G2/M and cell death in a dose-dependent manner as measured by the number of cells that exhibit sub-2n through 4n DNA content as revealed by DAPI staining. Measurements of cell death by forward and side scatter of light depicted in FIG. 3 corroborate this result and confirm that ON01910.Na induces cell death. FIG. 3 further illustrates that 24 and 48-hour periods of continuous ON01910.Na treatment induce cell death, with a greater magnitude of cell death observed following longer treatment and higher dose.

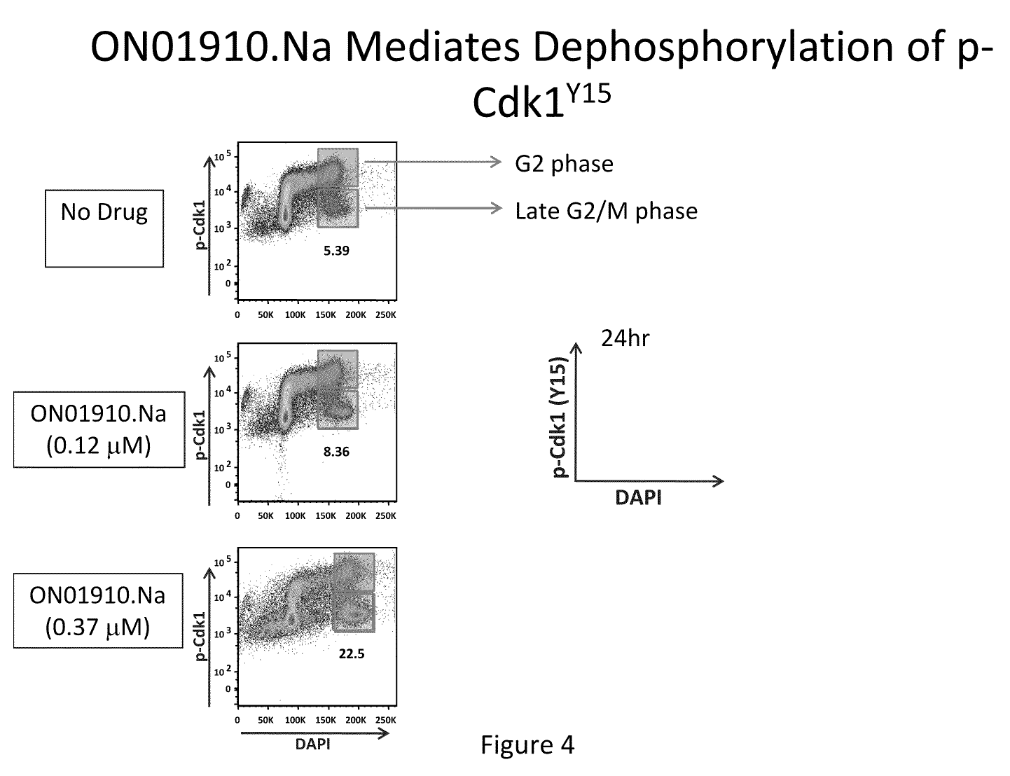

FIGS. 4-6 show the effects of two concentrations of ON01910.Na on different markers or nodes whose activation status correlates with cell cycle progression. FIG. 4 shows that 24 hours of continuous ON01910.Na treatment increases dephosphorylation of p-Cdk1 at tyrosine 15 which is necessary for activation. Cdk1 activation normally occurs at the end of G2, so increased Cdk1 levels dephosphorylated at tyrosine 15 are consistent with cell cycle arrest in G2 or early M phases (See Alberts et al, Molecular Biology of the Cell, 4th Ed., Chapter 17 for a detailed discussion of the cell cycle).

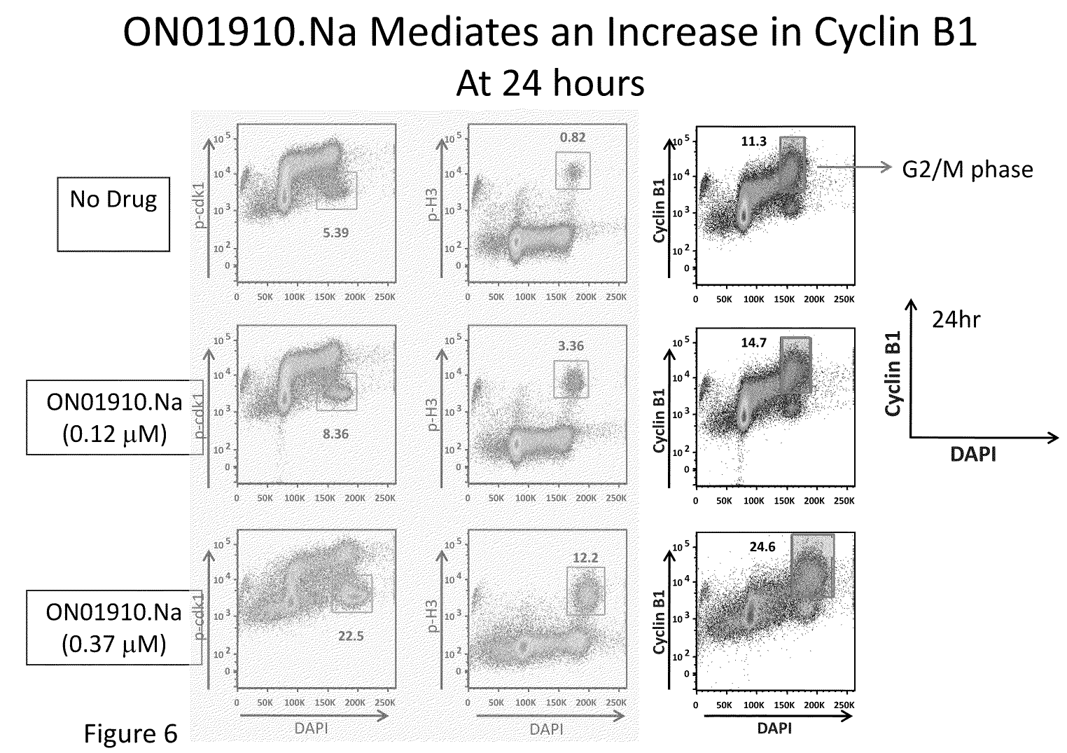

FIG. 5 shows that histone 3 serine 28 phosphorylation increases as TF-1 cells are treated with two increasing concentrations of ON01910.Na. This increase in histone 3 phosphorylation indicates that more cells enter M phase in a dose dependent manner following ON01910.Na treatment. Phosphorylation of histone H3 is a marker of chromatin condensation and entry into M phase (See Alberts et al, Molecular Biology of the Cell, 4th Ed., FIG. 4-35 for discussion of histone modifications). Continuous treatment with ON01910.Na for 24-hours enhances H3 (Ser28) phosphorylation. The effects of these two doses on the node state (serine 28 phosphorylation of H3) are detectable using flow cytometry. FIG. 6 shows that as TF-1 cells are exposed to an increased concentration of ON01910.Na, more cells express Cyclin B1. This result strongly suggests that ON01910.Na treatment induces arrest in the G2 or M phases of the cell cycle. FIG. 7 presents a summary of the effects of ON01910.Na treatment on the Cyclin B1, p-Cdk1 Y15, and pH3 S28 cell cycle nodes. All three nodes indicate that treatment with various concentrations of ON01910.Na for 24 hours induces TF-1 cells to arrest in the G2 or M phases of the cell cycle in a dose dependent manner.

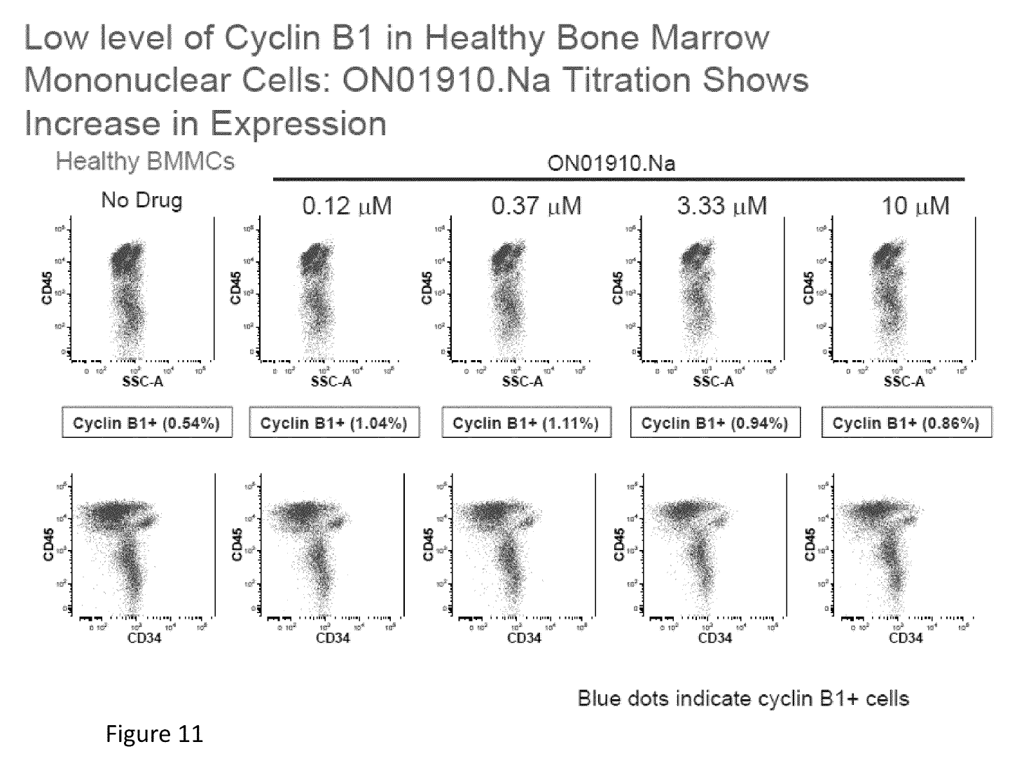

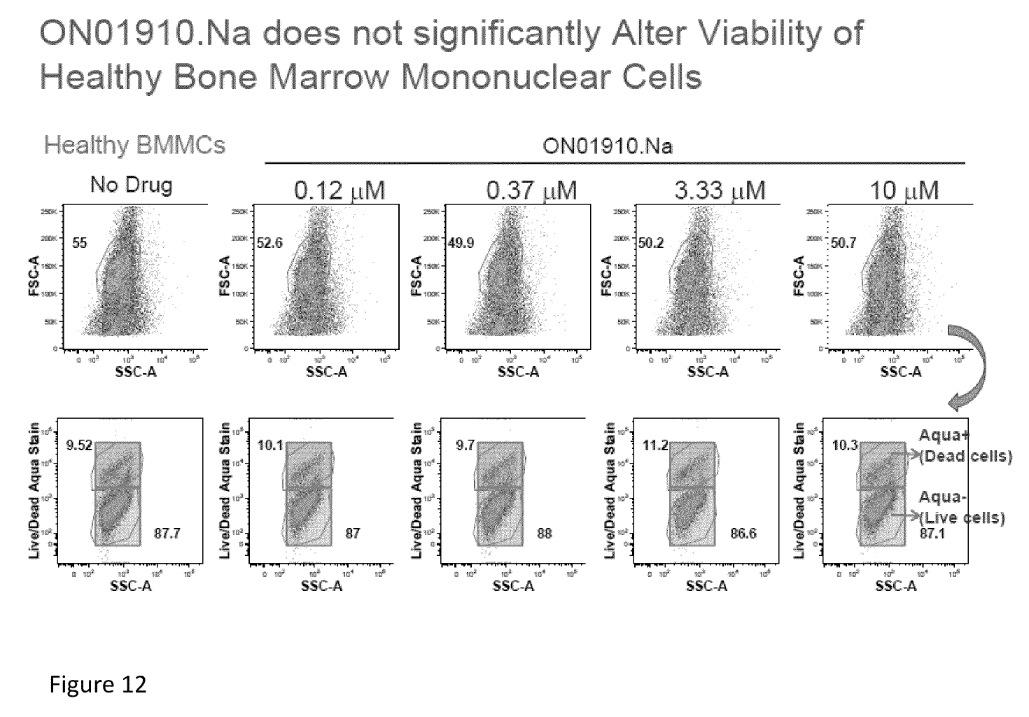

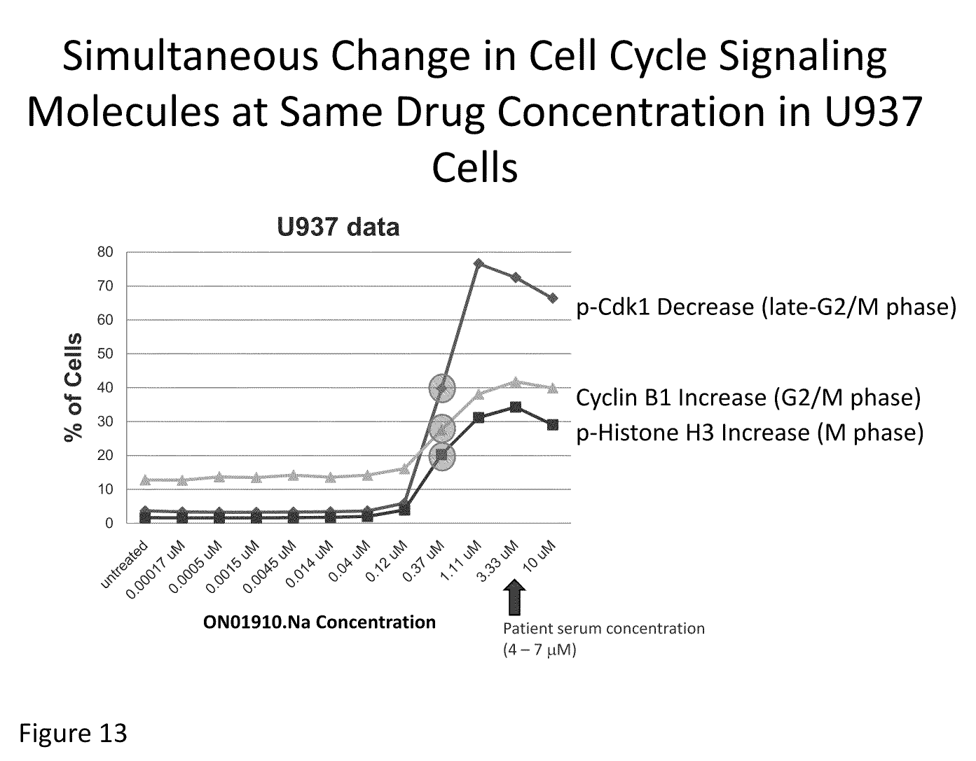

The findings shown in FIGS. 4 through 7 were tested in other cell types. A similar cell cycle arrest was observed in cells from the U937 cell line and in healthy bone marrow mononuclear cells (BMMCs). FIGS. 11 and 13 show decreased phosphorylation of Cdk1 and increased phosphorylation of H3 and expression of Cyclin B1 in response to increasing ON0190.Na titration in U937 cells (FIG. 13) after 24 hours of incubation. FIG. 11 demonstrates that Cyclin B1 expression levels increase in healthy BMMCs in response to ON01910.Na titration confirming the observations in TF-1 and U937 cultured human cells. FIG. 12 indicates that ON01910.Na titration does not significantly alter viability of healthy BMMCs.

This titration assay is an example of an embodiment of the invention useful for selecting drugs or combinations of drugs for specific diseases or individual patients, and/or for assessing dosing and schedule of drug treatment. One skilled in the art should appreciate that other cultured cell lines, cells types, modulators, and cell cycle nodes may be used without departing from the spirit of the invention.

### Example 2

FIGS. 9 and 10 show an embodiment of the invention used to analyze the effects of the DNA methyltransferase inhibitor drugs Vidaza® cytidine analog and Dacogen® cytidine analog on cultured U937 cells. See Example 1 in U.S. Ser. No. 61/120,320 for detailed cell culture, staining, and flow cytometry protocols similar to those used in this example. Here, to analyze the effects of Vidaza® cytidine analog and Dacogen® cytidine analog on cell cycle progression and cell death, U937 cells were treated with Vidaza® cytidine analog (5-Azacytidine) at a concentration of 2.5 μM, or Dacogen® cytidine analog (5-Aza-2′-deoxycytidine) at a concentration of 0.625 μM. The cells were continuously incubated with the drugs for 20 hours in 10% FBS RPMI, fixed, permeabilized, and then stained with DAPI, or stained with DAPI and immunolabeled with antibodies against DMNT1.

FIG. 9 shows that treatment with Vidaza® cytidine analog resulted in increased cell death (i.e. an increase in Sub-G1 cells shown in the DAPI frequency plots), while Dacogen® cytidine analog treatment resulted in S phase cell cycle arrest. Forward versus side scatter plots presented in FIG. 9 confirm these results. (See Alberts et al., Molecular Biology of the Cell, 4th Ed., Chapter 17 for an overview of the cell cycle). Only Vidaza® cytidine analog treatment decreased expression of the DNA methyltransferase DNMT1, which mediates maintenance DNA methylation (See FIG. 10) In this example, the invention can be used to predict whether a patient will respond to Vidaza® cytidine analog and/or Dacogen® cytidine analog. Additionally, the experimental results in this example may be used to identify therapeutic agents that can be used in combination with either Vidaza® cytidine analog or Dacogen® cytidine analog. For example, based on the result that Dacogen® cytidine analog increases the number of cells in S phase (See FIG. 9), an agent that drives cells into G2/M phase could be used in combination with Dacogen® cytidine analog to achieve an additive or synergistic effect with an agent designed to arrest cells another cell cycle phase, for example G1.

While preferred embodiments of the present invention have been shown and described in that application, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method for classifying a cell comprising:
contacting the cell with a targeted cell cycle pathway modulator;
determining the presence or absence of a change in activation level of an activatable element in the cell; and
classifying the cell based on the presence or absence of the change in the activation level of the activatable element.

2. The method of claim 1, wherein the change in activation level of the activatable element is an increase in activation level of the activatable element.

3. The method of claim 1, wherein the cell is a cancer cell.

4. The method of claim 1, wherein the activatable element is cyclin A, cyclin B, cyclin B1, Plk1, Histone H3, cyclin D, cyclin E, CDK1, CDK2, CDK3, CDK4, CDK5, CDK6, CDK7, CDK8, CDK9, CDK10, CDK11, CDK12, CDK13, Wee, CDK-activating kinase (CAK), Cdc20, Cdc25, retinoblastoma susceptibility protein (Rb), p21, p27, p57, p53, Tumor Growth Factor beta (TGFβ), p16INK4a, p14ARF, caspase-2, caspase-3, caspase-6, caspase-7, caspase-8, caspase-9, cytochrome c, Bcl-2, survivin, Xiap, PARP, Chk1, Chk2, histone 2AX, TRADD, FADD, Fas receptor, FasL, caspase-10, BAX, BID, BAK, BAD, Bcl-X_{L}, SMAC, VDAC2, Bim, Mcl-1 or AIF.

5. The method of claim 1, wherein the presence or absence of a change in the activation level of the activatable element is compared to a normal cell contacted with a targeted cell cycle pathway modulator.

6. The method of claim 1, wherein the targeted cell cycle modulator is a cell cycle inhibitor.

7. The method of claim 6, wherein the cell cycle inhibitor is an alkylating agent.

8. The method of claim 7, wherein the alkylating agent is altretamine, busulfan, carboplatin, chlorambucil, cisplatin, cyclophosphamide, dacarbazine, ifosfamide, lomustine, mechlorethamine, melphelan, or procarbazine.

9. The method of claim 6, wherein the cell cycle inhibitor is a product that causes DNA damage.

10. The method of claim 9, wherein the product that causes DNA damage is bleomycin, daunorubicin, docetaxel, doxorubicin, epirubicin, etoposide, homoharringtonine, idarubicin, irinotecan, mitomycin, mitoxantrone, paclitaxel, topotecan, vinblastine, vincristine, or vinorelbine.

11. The method of claim 6, wherein the cell cycle inhibitor is an antimetabolite.

12. The method of claim 11, wherein the antimetabolite is azacytidine, cladribine, cytarabine, floxuridine, fludarabine, fluorouracil, edatrexate, gemcitabine, hydroxyurea, mercaptopurine, methotrexate, pentostatin, thioguanine or tomudex.

13. The method of claim 1, wherein the presence or absence of a change in the activation levels of the activatable element is determined in the determining step.

14. The method of claim 1, wherein the classification comprises classifying the cell as a cell that is correlated with a clinical outcome.

15. The method of claim 14, wherein the clinical outcome is the presence or absence of a cancer, immune, autoimmune, diabetes, cardiovascular, metabolic disorder, degenerative/wasting, neurological, endocrine, or viral disorder.

16. The method of claim 14, wherein the clinical outcome is the staging or grading of a cancer condition.

17. The method of claim 1, wherein the classification further comprises determining a method of treatment.

18. The method of claim 1, wherein the modulator is a cancer cell modulator.

19. The method of claim 1, wherein the modulator is a growth factor, chemokine, cytokine, drug, immune modulator, ion, neurotransmitter, adhesion molecule, hormone, small molecule, inorganic compound, polynucleotide, antibody, natural compound, lectin, lactone, chemotherapeutic agent, biological response modifier, carbohydrate, protease, free radical, complex and undefined biologic composition, cellular secretion, glandular secretion, physiologic fluid, reactive oxygen species, virus, electromagnetic radiation, ultraviolet radiation, infrared radiation, particulate radiation, redox potential, pH modifier, the presence or absences of a nutrient, change in temperature, change in oxygen partial pressure, change in ion concentration or application of oxidative stress.

20. The method of claim 1, further comprising analyzing expression level of the cell cycle pathway protein.

21. The method of claim 20, wherein the cell is from a patient sample.

22. The method of claim 21, further comprising determining a clinical outcome based on the correlation of the activity of a cell cycle protein with the expression level of the cell cycle pathway protein.

23. The method of claim 22, further comprising determining a method of treatment of the patient based on the activity of the cell cycle pathway protein.

24. A method of determining the presence or absence of a condition in an individual comprising:
subjecting a cell from the individual to a targeted cell cycle pathway inhibitor;
determining the activation level of an activatable element in the cell; and
determining the presence or absence of the condition based on the activation level.

25. A method of correlating and/or classifying an activatable state of a cancer cell with a clinical outcome in an individual comprising:
subjecting the cancer cell from the individual to a targeted cell cycle pathway modulator;
determining the activation level of an activatable element; and
identifying a pattern of the activation level of the activatable element to determine the presence or absence of an alteration in signaling, wherein the presence of the alteration is indicative of a clinical outcome.

26. A method of analyzing the effect of a targeted cell cycle pathway compound comprising:
contacting a cell with the cell cycle pathway targeting compound and analyzing activity of a cell cycle pathway protein in said cell.

27. A method of ameliorating a cell cycle pathway disorder comprising:
administering to a subject a first compound;
determining the cell cycle phase of a cell from the subject from the activation level of an activatable element;
administering to the subject a second compound at a time where the cell is in a predetermined cell cycle phase.

28. A kit comprising a targeted cell cycle pathway modulator, a state-specific binding element and instructions for use.
